(19) United States
(12) Patent Application Publication
(10) Pub. No.: US 2010/0105749 A1
(43) Pub. Date: Apr. 29, 2010

(54) CHEMICAL COMPOUNDS, PHARMACEUTICAL COMPOSITIONS AND METHODS

(75) Inventors: Jon Loren Collins, Durham, NC (US); Jodi Maglich Goodwin, Durham, NC (US); Millard Hurst Lambert, III, Durham, NC (US)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: SMITHKLINE BEECHAM CORPORATION, Philadelphia, PA (US)

(21) Appl. No.: 12/525,230

(22) PCT Filed: Jan. 30, 2008
(86) PCT No.: PCT/US08/52376

§ 371 Date: Jul. 30, 2009

Related U.S. Application Data
(60) Provisional application No. 60/887,961, filed on Feb. 2, 2007.

(51) Int. Cl. A61K 31/4184; C07D 235/30; A61P 9/00

(52) U.S. Cl. 514/395; 548/307.4

(57) ABSTRACT

The present invention provides compounds of the formulae (I) and (II), and derivatives thereof, for the treatment of cardiovascular conditions

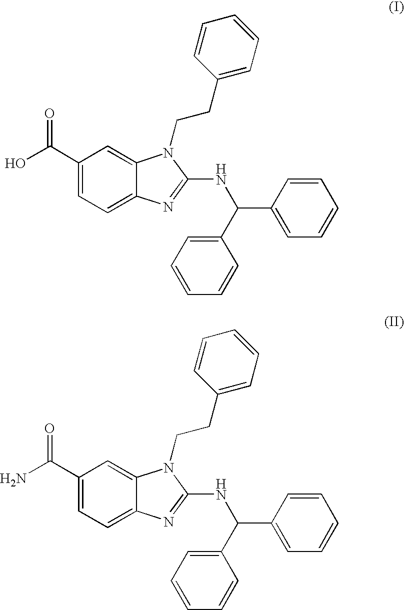

### TECHNICAL FIELD

The present invention relates generally to certain novel chemicals, pharmaceutical compositions and methods for the elevation of serum bilirubin and prevention or treatment of disease, particularly cardiovascular conditions, in humans.

### BACKGROUND

Low serum bilirubin concentrations are associated with an increased risk of coronary heart disease (CHD) as reported in many studies, see J. Lin in Circulation, Oct. 3, 2006, pages 1476-1481. In a prospective study of serum bilirubin and cardiovascular disease (CVD) in the Framingham Offspring Study, higher serum bilirubin concentrations were associated with decreased risk of CVD, CHD and myocardial infarction (MI), see L. Djousse in Am J Cardiology, 2001; 87; 1196-1200.

An unambiguous inverse relationship between serum bilirubin levels and atherosclerosis was demonstrated in a preliminary meta-analytical study by L Novotny in Exp Biol Med 228:568-571 (2003). The relationship between bilirubin concentrations and the constitutive androstane receptor (CAR) was explored by W Huang in PNAS, Apr. 1, 2003, Vol. 100, No. 7 at pages 4156-4161 and www.pnas.org. In an editorial in Clinical Chemistry 49, No. 7 at pages 1039-1040 (2003), H Schwertner explained the presence of an inverse relationship between serum bilirubin concentrations and coronary artery disease (CAD) incidence.

### SUMMARY OF THE INVENTION

In one embodiment, the compound 2-(benzhydrylamino)-1-(2-phenylethyl)-1H-benzimidazole-6-carboxylic acid, or a salt or ester thereof.

In one embodiment, novel pharmaceutical compositions for the prophylaxis or treatment of a cardiovascular disease. In one embodiment, a compound to be used in the pharmaceutical composition is 2-(benzhydrylamino)-1-(2-phenylethyl)-1H-benzimidazole-6-carboxylic acid, or a salt or ester thereof. In one embodiment, a compound to be used in the pharmaceutical composition is 2-(benzhydrylamino)-1-(2-phenylethyl)-1H-benzimidazole-6-carboxamide or a salt thereof.

### DETAILED DESCRIPTION OF THE INVENTION

It is to be understood that the terminology used herein is for the purpose of describing particular embodiments only, and is not intended to limit the scope of the present invention, the invention being defined by the claims.

Unless defined otherwise, all technical and scientific terms used herein are intended to have their ordinary meanings as understood by one of ordinary skill in the art to which this invention pertains. Although any methods and materials similar or equivalent to those described herein can be used in the practice or testing of the present invention, representative methods, devices, and materials are now described.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the present invention.

It is understood that the present invention compounds of formula (I) and compositions containing compounds of formulas (I) or (II) encompasses such compounds in the form of the depicted carboxylic acid of formula (I), the amide of formula (II) as well as the corresponding active moiety equivalents thereof including isomers, salts, tautomers, solvates and polymorphic crystalline structures. Such embodiments, including the esters of (I), may be noted herein, for example in describing pharmaceutical compositions, as simply compounds of formulae (I) and (II). In addition, it can be noted that esters themselves can also be salts and so the novel compounds of the invention and the compounds used in the novel compositions includes individual aspects, such as esters, as well as multiple facets of such compounds, eg ester salts in the form of a solvate.

The term “therapeutically effective amount” refers to that amount of a modulator, drug, or other molecule that is sufficient to effect treatment when administered to a subject in need of such treatment. The therapeutically effective amount will vary depending upon the subject and disease condition being treated, the weight and age of the subject, the severity of the disease condition, the manner of administration and the like, which can readily be determined by one of ordinary skill in the art.

The term “solvate” refers to a complex of variable stoichiometry formed by a solute (in this invention, a compound of formula I or a salt thereof) and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include water, ethanol and acetic acid. Most preferably the solvent used is water.

Typically, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts derived from a nitrogen on a substituent in a compound of the present invention. Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate, triethiodide, trimethylammonium and valerate. Other salts, which are not pharmaceutically acceptable, may be useful in the preparation of compounds of this invention and these form a further aspect of the invention.

In one embodiment, the present invention provides compounds of formula (I) and pharmaceutically acceptable salts or esters thereof:

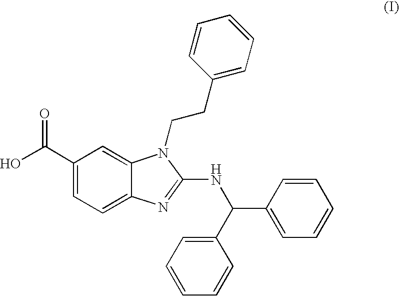

In a further aspect of the invention, pharmaceutical compositions are provided which may contain one or more of compounds of formula (I) or salts or esters thereof as well as compounds of formula (II) and salts thereof:

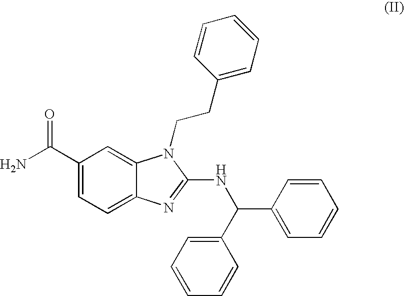

While it is possible that, for use in the prevention, delay in the onset of or treatment of cardiovascular conditions, a compound of formula (I) or (II) may be administered as the raw chemical, it is possible to present the active ingredient as a pharmaceutical composition. Accordingly, the invention further provides pharmaceutical compositions, which may be administered in the methods of treating cardiovascular diseases or disorders of the present invention. The pharmaceutical compositions include a compound of formula (I) or (II) or salt thereof, and one or more pharmaceutically acceptable carriers, diluents, or excipients. The carrier(s), diluent(s) or excipient(s) must be acceptable in the sense of being compatible with the other ingredients of the formulation and not deleterious to the recipient thereof.

For the prevention, delay of the onset, or treatment of cardiovascular conditions, the projected human dose of a compound of formula (I) or (II), optionally divided in 1 to 4 doses, is one that brings the patient to a higher level of normal bilirubin, e.g. within the normal bilirubin range as set forth in Example A. Thus, the dose would be about 6-60 mg/kg of body weight, particularly about 3-30 mg/kg administered 2×/day. For an average human of about 65 kg, the projected dose would be about 400-4000 mg/day. Pharmaceutical formulations may be presented in unit dose forms containing a predetermined amount of active ingredient per unit dose whereby a therapeutically effective amount of an active medicament is administered to a patient. Such a unit may contain, for example, about 100 to 1000 mg of a compound of formula (I) or (II) and may vary, along with the dose per day, with the bioavailability and water solubility of the particular compound of formula (I) or (II). The dose and unit dose would be chosen by the physician depending on the condition being treated, the route of administration and the age, weight and condition of the patient. Preferred unit dosage formulations are those containing a daily dose or sub-dose, as herein above recited, or an appropriate fraction thereof, of an active ingredient. Furthermore, such pharmaceutical formulations may be prepared by any of the methods well known in the pharmacy art.

The compound of formula (I) or (II) may be administered by any appropriate route. Suitable routes include oral, rectal, nasal, topical (including buccal and sublingual), vaginal, and parenteral (including subcutaneous, intramuscular, intraveneous, intradermal, intrathecal, and epidural). It will be appreciated that the preferred route may vary with, for example, the condition of the recipient.

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax or the like.

Syntheses of compounds used in the present inventions can be summarized in the following reaction scheme:

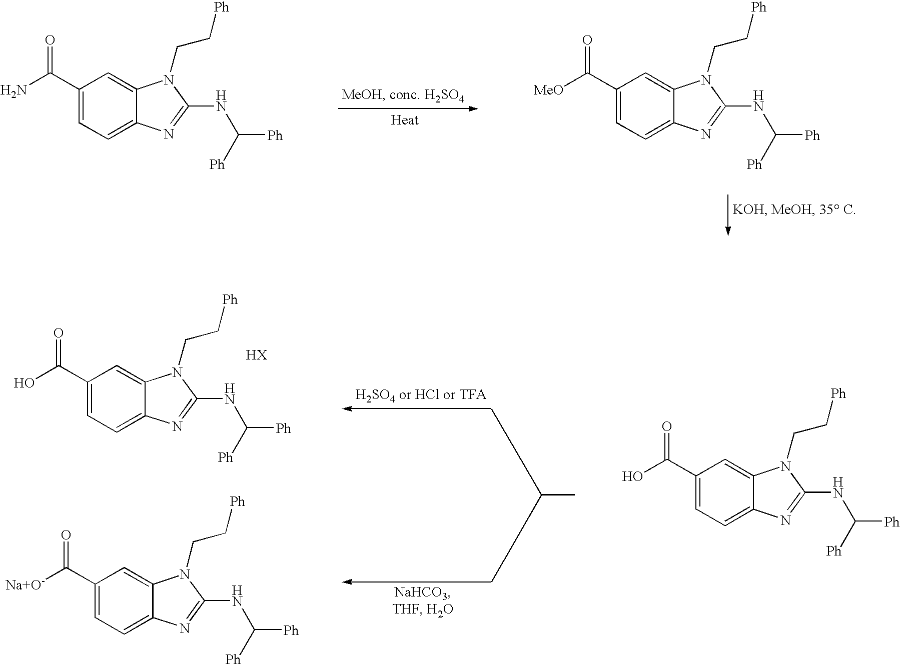

### EXAMPLES

The following Examples have been included to illustrate exemplary modes of the invention. Certain aspects of the following Examples are described in terms of techniques and procedures found or contemplated to work well in the practice of the invention. These Examples are exemplified through the use of standard laboratory practices of the inventors. In light of the present disclosure and the general level of skill in the art, those of skill will appreciate that the following Examples are intended to be exemplary only and that numerous changes, modifications, and alterations can be employed without departing from the spirit and scope of the invention. ¹H-NMR spectra were recorded on a Varian Gemini 400 MHz NMR spectrometer. ¹H-NMR spectra are reported as chemical shift, number of protons, multiplicity (s, singlet; d, doublet; t, triplet; m, multiplet; br s, broad singlet) and coupling constant (J) in Hertz. Electron Spray (ES) or Chemical Ionization (CI) was recorded on a Hewlett Packard 5989A mass spectrometer. Abbreviations include: MeOH (methanol); EtOAc (ethyl acetate); HPLC (high pressure liquid chromatography); THF (tetrahydrofuran); TFA (trifluoroacetic acid); M (molar); Et₂O (diethyl ether); Ph (phenyl); SBE-CD (sulfobutylether-β-cyclodextrin); DMSO (dimethyl sulfoxide); AST (Aspartate aminotransferase); and ALT (Alanine transaminase).

### Example 1

### Methyl 2-[(diphenylmethyl)amino]-1-(2-phenylethyl)-1H-benzimidazole-6-carboxylate trifluoroacetate

### Formula (I): Methyl Ester, Trifluoroacetate Salt

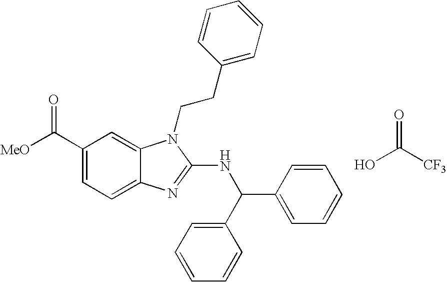

To a solution of 30 mg (0.06 mmol) of 2-[(diphenylmethyl)amino]-1-(2-phenylethyl)-1H-benzimidazole-6-carboxamide, the product of Example 6, in 3 mL MeOH was added 1 mL conc. H₂SO₄ slowly. The reaction was shaken overnight at 60° C. The reaction was cooled to room temperature, quenched with sat. NaHCO₃, and extracted with EtOAc. The organic phase was dried with MgSO₄ and concentrated on a rotary evaporator. Purification by reverse phase HPLC (10-100% aqueous CH₃CN with 0.05% TFA) provided 8 mg (26%) of the title compound 1: ¹H NMR (400 MHz, CDCl₃) δ ppm 3.07 (t, J=6.51 Hz, 2H) 3.93 (s, 3H) 4.36 (t, J=6.60 Hz, 2H) 6.26 (d, J=7.31 Hz, 1H) 6.96-7.00 (m, 2H) 7.10-7.21 (m, 13H) 7.45 (d, J=8.03 Hz, 1H) 7.51 (br s, 1H) 7.72 (d, J=1.07 Hz, 1H) 7.89 (dd, J=8.47, 1.52 Hz, 1H); mass spectrum (ES) m/e=462 (M+1).

### Example 2

### 2-[(diphenylmethyl)amino]-1-(2-phenylethyl)-1H-benzimidazole-6-carboxylic acid trifluoroacetate

### Formula (I): Trifluoroacetate Salt

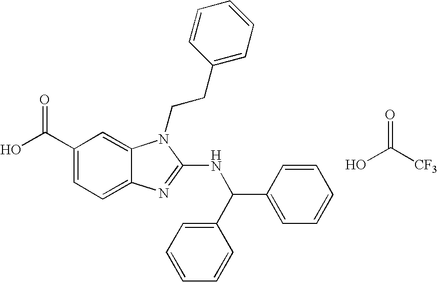

To a solution of 50 mg (0.108 mmol) of methyl 2-[(diphenylmethyl)amino]-1-(2-phenylethyl)-1H-benzimidazole-6-carboxylate trifluoroacetate, the product of Example 1, in 0.3 mL MeOH was added 18 mg (0.324 mmol) of potassium hydroxide. The reaction was loosely capped and stirred overnight at 35° C. The solvent evaporated overnight so another 1 mL of MeOH and 36 mg of potassium hydroxide was added to the reaction. After 6 hours the reaction was diluted with 3 mL of MeOH and 10 mL of H₂O, acidified with 1 M H₂SO₄ and extracted with EtOAc. The organic solvent was dried with MgSO₄ and concentrated by rotary evaporator. Purification by reverse phase HPLC (10-100% aqueous CH₃CN with 0.05% TFA) provided 22 mg (46%) of the title compound 2: ¹H NMR (400 MHz, MeOH-d₄) δ ppm 3.14 (t, J=6.51 Hz, 2H) 4.62 (t, J=6.60 Hz, 2H) 6.06 (s, 1H) 6.98-7.04 (m, 2H) 7.13-7.19 (m, 3H) 7.21-7.26 (m, 4H) 7.34-7.45 (m, 7H) 7.87-7.90 (m, 1H) 7.96 (dd, J=8.38, 1.61 Hz, 1H); mass spectrum (ES) m/e=448 (M+1).

### Example 3

### 2-[(diphenylmethyl)amino]-1-(2-phenylethyl)-1H-benzimidazole-6-carboxylic acid hydrochloride

### Formula (I); Hydrochloride Salt

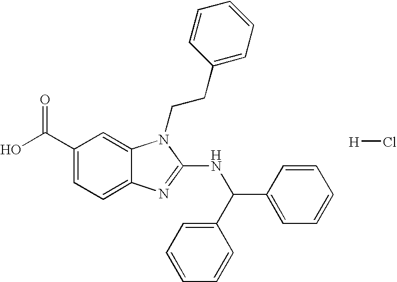

Approximately 50 mg of 2-[(diphenylmethyl)amino]-1-(2-phenylethyl)-1H-benzimidazole-6-carboxylic acid trifluoroacetate, the product of Example 2, was added to a vial and dissolved in MeOH/EtOAc. This solution was washed with sat. NaHCO₃, H₂O, and brine. The organic phase was dried with MgSO₄ and concentrated by rotary evaporator. To the subsequent amorphous solid was added 3 mL of 1 M HCl in Et₂O and stirred until solid formed. The solvent was removed by rotary evaporator to afford 21 mg of the title compound 3: ¹H NMR (400 MHz, MeOH-d₄) δ ppm 3.14 (t, J=6.51 Hz, 2H) 4.62 (t, J=6.33 Hz, 2H) 6.04 (s, 1H) 7.00-7.04 (m, 2H) 7.15-7.20 (m, 3 H) 7.20-7.25 (m, 4H) 7.34-7.45 (m, 7H) 7.88-7.90 (m, 1H) 7.96 (dd, J=8.38, 1.43 Hz, 1H); mass spectrum (ES) m/e=448 (M+1).

### Example 4

### 2-[(diphenylmethyl)amino]-1-(2-phenylethyl)-1H-benzimidazole-6-carboxylic acid sulfate

### Formula (I): sulfate salt

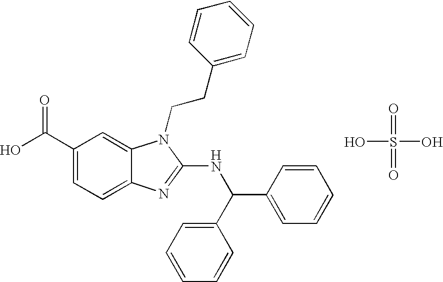

Approximately 50 mg of 2-[(diphenylmethyl)amino]-1-(2-phenylethyl)-1H-benzimidazole-6-carboxylic acid trifluoroacetate, the product of Example 2, was added to a vial and dissolved in MeOH/EtOAc. This solution was washed with sat. NaHCO₃, H₂O, and brine. The organic phase was dried with MgSO₄ and concentrated by rotary evaporator. To the subsequent amorphous solid was added Et₂O and EtOAc until the solid dissolved followed by a few drops of conc. H₂SO₄. The solution was stirred until white precipitate formed. The solution was filtered and collected solid was washed with EtOAc. The salt was dried on vacuum filter to afford 20 mg of the title compound 4: ¹H NMR (400 MHz, MeOH-d₄) δ ppm 3.14 (t, J=6.42 Hz, 2H) 4.62 (t, J=6.60 Hz, 2H) 6.03 (s, 1H) 7.00-7.04 (m, 2H) 7.15-7.20 (m, 3H) 7.20-7.25 (m, 4H) 7.34-7.45 (m, 7H) 7.90 (d, J=1.25 Hz, 1H) 7.97 (dd, J=8.38, 1.43 Hz, 1H); mass spectrum (ES) m/e=448 (M+1).

### Example 5

### 2-[(diphenylmethyl)amino]-1-(2-phenylethyl)-1H-benzimidazole-6-carboxylic acid sodium salt

### Formula (I): sodium salt

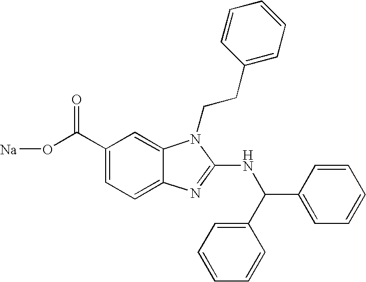

A solution of 58 mg (0.13 mmol) of 2-[(diphenylmethyl)amino]-1-(2-phenylethyl)-1H-benzimidazole-6-carboxylic acid trifluoroacetate, the product of Example 2, in MeOH/EtOAc was washed with sat. NaHCO₃, H₂O, and brine. The organic phase was dried with MgSO₄ and concentrated by rotary evaporator. The subsequent resin was dissolved in 5 mL of THF and 0.259 mL of a 0.5 M solution of NaHCO₃ was added via pipet. Some solid precipitated out of solution and was dissolved with the addition of a small amount of H₂O. The solution was concentrated by rotary evaporator to afford 52 mg of the title compound 5: ¹H NMR (400 MHz, MeOH-d₄) δ ppm 3.08 (t, J=6.96 Hz, 2H) 4.38 (t, J=6.96 Hz, 2H) 6.15 (s, 1H) 7.06-7.18 (m, 6H) 7.18-7.26 (m, 7H) 7.27-7.32 (m, 4H) 7.75 (dd, J=8.29, 1.52 Hz, 1H) 7.85 (d, J=1.07 Hz, 1H); mass spectrum (ES) m/e=448 (M+1).

### Example 6

### 2-(benzhydrylamino)-1-(2-phenylethyl)-1H-benzimidazole-6-carboxamide

### Formula (II)

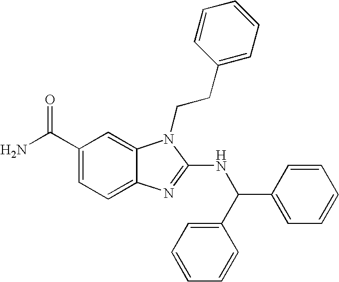

2-(benzhydrylamino)-1-(2-phenylethyl)-1H-benzimidazole-6-carboxamide (also known as 2-[(diphenylmethyl)amino]-1-(2-phenylethyl)-1H-benzimidazole-6-carboxamide) of Formula (II) was synthesized as follows. A solution of 3-fluoro-4-nitrobenzoic acid (1.28 g; 6.9 mmol) in 10 mL anhydrous N,N-dimethylformamide was treated with [O-(7-azabenzotriazol-1-yl)-1,1,3,3-tetramethyluronium hexafluoro-phosphate] (2.6 g; 6.9 mmol) followed by N,N-diisopropylethylamine (3.6 ml, 20.7 mmol). After shaking for 5 min, the mixture was added to polystyrene Rink amide AM resin (1.0 g; 0.69 mmol/g; 0.69 mmol), and the reaction was rotated at 25° C. for 18 h. The reaction solution was drained, and the resin was washed sequentially with N,N-dimethylformamide (3×), dichloromethane (3×), methanol (2×), and dichloromethane (3×). The dried resin was treated with 15.2 ml of a 0.5 M phenethylamine in N-methylpyrrolidinone solution then rotated at 70° C. for 15 hours. The cooled reaction was drained, and the resin was washed sequentially with N,N-dimethylformamide (3×), dichloromethane (3×), methanol (2×), and dichloromethane (3×). The resin was treated with 3.8 ml of 2.0 M 5 nCl₂.dihydrate in N-methylpyrrolidinone solution and rotated at 25° C. for 24 hours. The reaction was drained and the resin washed sequentially with 30% ethylenediamine (3×), N,N-dimethylformamide (3×), dichloromethane (3×), methanol (2×), and dichloromethane (3×). The dried diamine resin was treated with 7.6 ml of a 0.5 M benzyhydryl isothiocyanate in N-methylpyrrolidinone solution and 7.6 ml of a 1.0 M diisopropylcarbodiimide in N-methylpyrrolidinone solution. After rotating at 80° C. for 24 h the reaction was cooled to 25° C., drained, and the resin was washed sequentially with N,N-dimethylformamide (3×), dichloromethane (3×), methanol (2×), and dichloromethane (3×). The resin was treated with 30 ml 95% trifluoroacetic acid (TFA) in water and rotated at 25° C. for 3 hours. The resin was drained and washed with dichloromethane. The filtrate was concentrated in vacuo to give an oil. The oil was redissolved in dichloromethane and the solution was washed twice with saturated sodium bicarbonate (NaHCO₃). The organic layer was dried (Na₂SO₄), filtered, and concentrated in vacuo. The crude product was triturated with Et₂O/hexanes, and the solid was collected by filtration to give 333 mg (98% yield) of the title compound 6 as an off-white solid: ¹H NMR (DMSO-d₆, 400 MHz) δ 7.68 (m, 2H), 7.63 (d, 1H, J=8.4), 7.54 (dd, 1H, J=8.0, 1.2), 7.40-7.00 (m, 17H), 6.36 (d, 1H, J=8), 4.42 (t, 2H, J=7.4), 2.97 (t, 2H, J=7.4); MS (ESP+) m/e 447 (MH⁺).

### Example A

### Bilirubin Monkey Test

The product of Example 4 was formulated in 5% DMSO: 2% Tween 80: 10% SBE-CD with 1 molar equiv of HCl at 6 mg/ml and dosed at 30 mg/kg. Cynomolgus monkeys (each group n=6) had baseline blood drawn at day 0 at 9 am. On day 1, the monkeys were fed their morning meal at 7 am. After 1 hour, all left over food was removed and fast was begun. There was twice a day oral dosing of compound or vehicle on day 1 and day 2 of the fast, on day 3 there was an oral dose the morning (8 am) before the blood draw (9 am). Food was returned to the monkeys at this point. Oral dosing occurred at approximately 8:00 a.m. and 4:00 p.m. administered by oral gavage. Serum chemistries were screened on baseline and end of treatment blood draws. Significant changes were detected in bilirubin levels. Bilirubin increased in the fasted state, as expected (total, direct, and indirect bilirubin changed 2.0-, 2.4- and 1.92-fold, respectively over baseline). Dosing of the product of Example 4 caused further increases in bilirubin levels (total, direct, and indirect bilirubin changed 3.74-, 4.71-fold, and 3.53-fold respectively over baseline).

### Bilirubin Values:

| | TBIL mg/dL | DBIL mg/dL | IBIL mg/dL |
|---|---|---|---|
| Baseline | 0.195 +/− 0.009 | 0.035 +/− 0.003 | 0.16 +/− .007 |
| 48 hr fast-Vehicle | 0.392 +/− 0.04 | 0.083 +/− 0.01 | 0.308 +/− .034 |
| 48 hr fast- | 0.73 +/− 0.04 | 0.165 +/− 0.01 | 0.565 +/− .036 |
| Compound 4 | | | |

Statistical analysis of people with high normal or slightly elevated bilirubin levels in blood shows that they have a lower risk of developing cardiovascular diseases.

Normal Clinical Values:

DBIL: direct bilirubin: 0.1 to 0.3 mg/dl

TBIL: total bilirubin: 0.3 to 1.5 mg/dl

IBIL: Indirect bilirubin: 0.2-0.7 mg/dl

No increases were detected in serum ALT or AST levels with drug treatment.

It will be understood that various details of the invention can be changed without departing from the scope of the invention. Furthermore, the foregoing description is for the purpose of illustration only, and not for the purpose of limitation, the invention being defined by the claims.

1. A compound of the following formula (I) 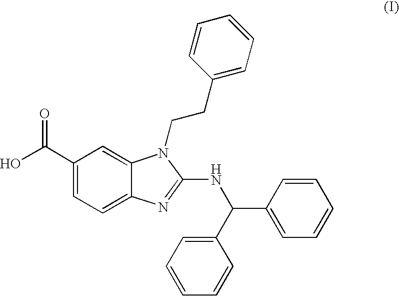
or a pharmaceutically acceptable salt thereof.

2. The compound of claim 1, wherein said compound is of the formula (I).

3. The compound of claim 1, wherein said compound is a pharmaceutically acceptable salt of formula (I).

4. (canceled)

5. A pharmaceutical composition comprising a compound according to claim 1 and a pharmaceutically acceptable carrier.

6. A method of treating cardiovascular disease in a human comprising administering to the human the pharmaceutical composition of claim 5.
